(19) United States
(12) Patent Application Publication
(10) Pub. No.: US 2010/0298953 A1
(43) Pub. Date: Nov. 25, 2010

(54) DEVICE AND METHOD FOR POSITIONING A SURGICAL PROSTHESIS

(75) Inventor: Michael David Holzman, Franklin, TN (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: VANDERBILT UNIVERSITY, Nashville, TN (US)

(21) Appl. No.: 12/740,553

(22) PCT Filed: Oct. 30, 2008
(86) PCT No.: PCT/US08/81790

§ 371 Date: Aug. 5, 2010

Related U.S. Application Data
(60) Provisional application No. 60/984,107, filed on Oct. 31, 2007.

(51) Int. Cl. A61F 2/02

(52) U.S. Cl. 623/23.72

(57) ABSTRACT

In one aspect, the present invention relates to a medical device for positioning a surgical prosthesis on a fascial defect of a tissue wall. In one embodiment, the medical device has a removable flexible member having a body portion having at least one magnetic section, and a magnet member positioned in relation to the at least one magnetic section such that a movement of the magnet member causes the flexible member to move accordingly. The surgical prosthesis is detachably attachable to the flexible member, and the flexible member is introducible into and removable from a body cavity.

### CROSS-REFERENCE TO RELATED PATENT APPLICATION

This application claims priority to and the benefit of, pursuant to 35 U.S.C. §119(e), U.S. provisional patent application Ser. No. 60/984,107, filed Oct. 31, 2007, entitled “Device and Method for Placement and Positioning of a Surgical Prosthesis,” by Michael David Holzman, the content of which is incorporated herein in its entirety by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference were individually incorporated by reference.

This application is being filed as PCT International Patent Application in the name of Vanderbilt University, a U.S. National Corporation, applicant for all countries except the U.S., and Michael David Holzman, a U.S. resident, applicant for the designation of the U.S. only, on 31 October 2008.

### FIELD OF THE INVENTION

The present invention generally relates to a medical device, and more particularly, to a medical device that utilizes a flexible member and a magnet member to position a surgical prosthesis on a fascial defect of the abdominal wall and methods of implementing same.

### BACKGROUND OF THE INVENTION

Implantable mesh patches for the repair of inguinal and other abdominal wall hernias are well known. Typically, these patches are intended for permanent placement within a patient's body space. For example, it is understood that U.S. Pat. No. 5,824,082 to Brown discloses a prosthesis for use in hernia repair surgery having a preformed prosthetic fabric supported along its periphery by a shape memory alloy wire having a transformation temperature corresponding to normal body temperature, allowing the prosthesis to be tightly rolled into a cylindrical configuration for delivery. Also, it is understand that U.S. Pub. No. 20070265710 to Brown et al. describes a hernia patch comprising a frame made of a plurality of strands made from a shape memory alloy wound together as a cable and a prosthetic mesh material attached to the cable frame. The cable frame forms a loop of a predetermined shape when unconstrained. The cable frame cam is rolled or folded into a tight cylindrical shape and inserted into a small diameter trocar. When the hernia patch is ejected out of the trocar into the patient's abdominal cavity, the frame warms to the point where the alloy is in its austenite form so that it springs to a functional, predetermined configuration. The frame is integral with the prosthetic mesh material so that it will not migrate and therefore will not need to be sutured or stapled in place.

Additionally, it is understand that U.S. Pat. No. 6,241,768 to Agarwal et al. discloses a prosthesis device for repairing a hernia having an insertion canal made of sheet material for extending through the hernia. The insertion canal is attached at one end to a collar and at the other end to a base that is to be on the inside of the abdominal cavity. The base may be a sheet or a pouch that is to be brought to bear on the inside the wall of the abdominal cavity. The insertion canal provides a simple way of deploying the sheet or pouch against the wall of a abdominal cavity.

However, these existing medical devices do not have a structure to allow that ensures a medical professional performing surgery to accurately position a surgical prosthesis to a specific site of a fascial defect for repairing the defect, without the need for a large number of surgical instruments and other complications at the site of the repair.

Therefore, a heretofore unaddressed need still exists in the art to address the aforementioned deficiencies and inadequacies.

### SUMMARY OF THE INVENTION

In one aspect, the present invention relates to a medical device for positioning a surgical prosthesis on a fascial defect of a tissue wall of a living subject for repairing the fascial defect thereon. The tissue wall defines a body cavity. The fascial defect is a hernia or the like.

In one embodiment, the medical device has a flexible member having a first end, a second end and a body portion defined there between, the body portion having at least one magnetic section, and a magnet member positioned in relation to the at least one magnetic section such that a movement of the magnet member causes the flexible member to move accordingly. The surgical prosthesis is detachably attachable to the flexible member, and the flexible member is introducible into and removable from the body cavity. In one embodiment, the at least one magnetic section continuously extends through the length of the body portion of the flexible member.

In operation, the surgical prosthesis detachably attached to the flexible member is introduced into the body cavity, and the magnet member is positioned outside the body cavity to manipulate the flexible member to move so as to position the surgical prosthesis on the fascial defect of the tissue wall.

In one embodiment, the flexible member is formed in a ring shape configuration with a biocompatible material having a shape memory property such that when constrained, the flexible member is deformed in a pre-determined shape configuration that is substantially different from the ring shape configuration, and when unconstrained, the flexible member returns to the ring shape configuration. The biocompatible material comprises a conductive material including metal or polymer.

In one embodiment, the flexible member is placed without a surgical prosthesis and has the means for determining a location, and a size of the fascial defect.

The surgical prosthesis has a size that is larger than a size of the fascial defect. The surgical prosthesis is formed of a biocompatible mesh material, and the biocompatible mesh material comprises a tubular surgical mesh, a skirted surgical mesh, or a biological graft tissue.

In one embodiment, the surgical prosthesis is formed of the skirted surgical mesh having an outer mesh portion and an inner mesh portion, and the flexible member passes into a pocket defined between the outer mesh portion and the inner mesh portion.

In another embodiment, the surgical prosthesis is formed of the tubular surgical mesh in a roll having an opening that defines an inner diameter of the roll, and the flexible member passes through the opening and into the inner diameter of the roll.

In an alternative embodiment, the surgical prosthesis has a peripheral edge portion defining a tunnel therewith, and the surgical prosthesis is detachably attached to the flexible member through the tunnel.

In another aspect, the present invention relates to a method for repairing a fascial defect in a tissue wall of a living subject, where the tissue wall defines a body cavity. In one embodiment, the method includes the step of providing a medical device. The medical device includes a flexible member having a body portion having at least one magnetic section, and a magnet member positioned in relation to the at least one magnetic section such that a movement of the magnet member causes the flexible member to move accordingly. In one embodiment, the flexible member is formed in a ring shape configuration with a biocompatible material having a shape memory property such that when constrained, the flexible member is deformed in a pre-determined shape configuration that is substantially different from the ring shape configuration, and when unconstrained, the flexible member returns to the ring shape configuration. The flexible member may have means for determining a location and a size of the fascial defect.

Furthermore, the method includes the step of determining a location and a size of the fascial defect using the medical device. In one embodiment, the determining step comprises the steps of introducing the flexible member of the medical device into the body cavity, positioning the magnet member of the medical device outside the body cavity, adjusting the position of the magnet member of the medical device so as to move the flexible member to determine a location and a size of the fascial defect, marking the determined location of the fascial defect in the tissue wall, and removing the flexible member of the medical device from the body cavity. In one embodiment, the step of introducing the flexible member into the body cavity comprises the steps of straightening the flexible member, and introducing the straightened flexible member into the body cavity using a laparoscopic access device (trocar) or an endoscopic access device.

Moreover, the method includes the steps of providing a surgical prosthesis having a size that is larger than the size of the fascial defect, introducing the surgical prosthesis into the body cavity, introducing the flexible member into the body cavity, attaching the surgical prosthesis to the flexible member of the medical device in the body cavity, and positioning the surgical prosthesis on the fascial defect of the tissue wall by moving the magnet member around to cause the flexible member (with the surgical prosthesis attached) to move accordingly.

The surgical prosthesis is formed of a biocompatible mesh material, and wherein the biocompatible mesh material comprises a tubular surgical mesh, a skirted surgical mesh, or a biological graft tissue. In one embodiment, the surgical prosthesis is formed of the skirted surgical mesh having an outer mesh portion and an inner mesh portion, and the step of attaching the surgical prosthesis to the flexible member of the medical device comprises the step of passing the flexible member into a pocket defined between the outer mesh portion and the inner mesh portion. In another embodiment, the surgical prosthesis is formed of the tubular surgical mesh is formed in a roll having an opening that defines an inner diameter of the roll, and the step of attaching the surgical prosthesis to the flexible member of the medical device comprises the step of passing the flexible member through the opening and into the inner diameter of the roll. In an alternative embodiment, the surgical prosthesis has a peripheral edge portion defining a tunnel therewith, and the step of attaching the surgical prosthesis to the flexible member of the medical device comprises the step of passing the flexible member through the tunnel.

In one embodiment, the step of attaching the surgical prosthesis to the flexible member of the medical device in the body cavity comprises the step of expanding the flexible member to stretch the surgical prosthesis into a flat configuration corresponding to the size of the fascial defect.

The step of positioning the surgical prosthesis comprises the steps of adjusting the position of the magnet member of the medical device so as to align the flexible member to the marked location of the fascial defect in the tissue wall, fixing the surgical prosthesis to the tissue wall for repairing the fascial defect, detaching the flexible member of the medical device from the surgical prosthesis, and removing the flexible member of the medical device from the body cavity through the afore mentioned laparoscopic access device (trocar) or an endoscopic access device.

In one embodiment, the step of fixing the surgical prosthesis to the tissue wall is performed by a fixation device and/or suture.

In yet another aspect, the present invention relates to a method for repairing a fascial defect in a tissue wall of a living subject, where the tissue wall defines a body cavity.

In one embodiment, the method include comprising the steps of providing a flexible member having at least one magnetic section, and a magnet member positioned in relation to the at least one magnetic section such that a movement of the magnet member causes the flexible member to move accordingly, determining a location and a size of the fascial defect using the flexible member and the magnet member, providing a surgical prosthesis having a size that is larger than the size of the fascial defect, attaching the surgical prosthesis to the flexible member, introducing the flexible member with the attached surgical prosthesis into the body cavity, aligning the flexible member with the attached surgical prosthesis to the determined location of the fascial defect in the tissue wall, and fixing the surgical prosthesis to the tissue wall for repairing the fascial defect.

The method further includes the steps of detaching the flexible member from the surgical prosthesis, and removing the flexible member from the body cavity.

In one embodiment, the flexible member is formed in a ring shape configuration with a biocompatible material having a shape memory property such that when constrained, the flexible member is deformed in a pre-determined shape configuration that is substantially different from the ring shape configuration, and when unconstrained, the flexible member returns to the ring shape configuration. The flexible member may also have means for determining a location and a size of the fascial defect.

In one embodiment, the step of introducing the flexible member into the body cavity is performed by a laparoscopic access device or a endoscopic access device.

The determining step comprises the steps of introducing the flexible member into the body cavity, positioning the magnet member outside the body cavity, adjusting the position of the magnet member so as to move the flexible member to determine a location and a size of the fascial defect, marking the determined location of the fascial defect in the tissue wall, and removing the flexible member from the body cavity.

In a further aspect, the present invention relates to a medical device for repairing the fascial defect. In one embodiment, the medical device has a flexible member having a first end, a second end and a body portion defined therebetween, the body portion having at least one magnetic section, a surgical prosthesis detachably attached to the flexible member, and a magnet member positioned in relation to the at least one magnetic section such that a movement of the magnet member causes the flexible member with the attached surgical prosthesis to move accordingly to allow the surgical prosthesis to be placed on the fascial defect.

In yet a further aspect, the present invention relates to a kit comprising a medical device as disclosed above.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment, and wherein:

FIG. 1 shows schematically a flexible member of a medical device according to one embodiment of the present invention;

FIG. 2 shows schematically a flexible member of a medical device according to another embodiment of the present invention;

FIG. 3 shows schematically fascial defect repair using a medical device according to one embodiment of the present invention, where arrows indicate a path for placing and positioning a flexible member with a surgical prosthesis to the fascial defect on a tissue wall, using a magnet member indicated by dashed lines;

FIG. 4 shows schematically a surgical prosthesis attached to a flexible member placed on a fascial defect on a tissue wall;

FIG. 5 shows schematically a cross-sectional view of a flexible member with an attached surgical prosthesis according to one embodiment of the present invention;

FIG. 6 shows schematically a cross-sectional view of a flexible member with an attached surgical prosthesis according to another embodiment of the present invention;

FIG. 7 shows schematically a cross-sectional view of a flexible member with an attached surgical prosthesis according to yet another embodiment of the present invention;

FIG. 8 shows schematically a flexible member introduced into a body cavity using an laparoscopic access device (torcar) according to one embodiment of the present invention;

FIG. 9 shows a flowchart relating to a method for positioning surgical prosthesis on a fascial defect in a tissue wall according to one embodiment of the present invention; and

FIG. 10 shows a flowchart relating to a method for positioning surgical prosthesis on a fascial defect in a tissue wall according to another embodiment of the present invention.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. The use of examples anywhere in this specification, including examples of any terms discussed herein, is illustrative only, and in no way limits the scope and meaning of the invention or of any exemplified term. Likewise, the invention is not limited to various embodiments given in this specification.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

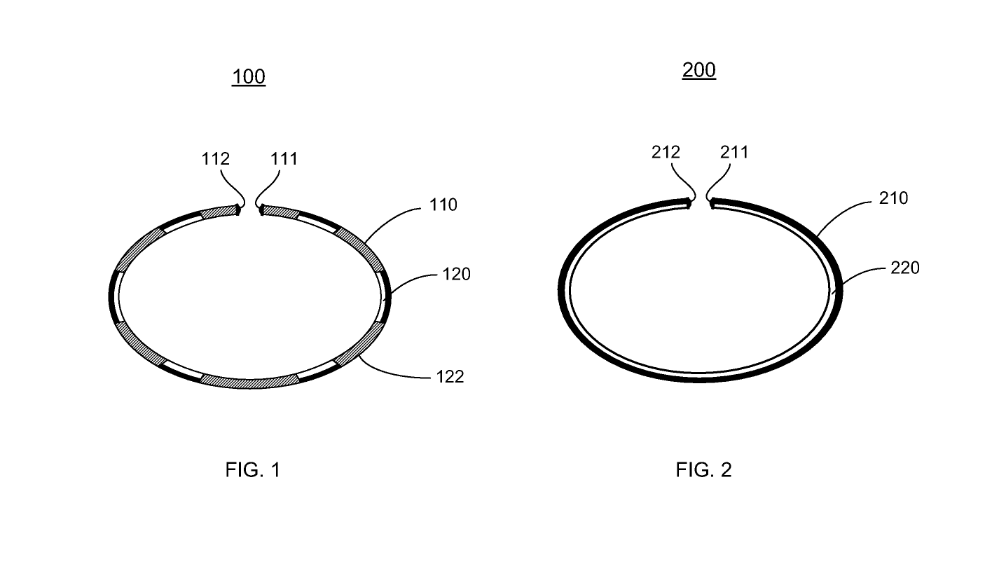

The description will be made as to the embodiments of the present invention in conjunction with the accompanying drawings in FIGS. 1-10. In accordance with the purposes of this invention, as embodied and broadly described herein, this invention, in one aspect, relates to a medical device for positioning a surgical prosthesis on a fascial defect of a tissue wall of a living subject for repairing the fascial defect (hernia) thereon. The tissue wall defines a body cavity. For example, the tissue wall is an abdominal wall and the body cavity is corresponding to an abdominal cavity. The fascial defect can be an abdominal wall hernia or others. The living subject can be a human or an animal.

The medical device has a flexible member having one or more one magnetic sections and a magnet member positioned in relation to the one or more one magnetic sections such that a movement of the magnet member causes the flexible member to move accordingly. In practice, the flexible member is introduced into the body cavity for determining the location and size of the fascial defect, and for positioning a surgical prosthesis on the determined location. After the surgical prosthesis is fixed to the tissue wall at the determined location for repairing the fascial defect thereon, the flexible member can be removed from the body cavity.

Referring FIG. 1, in one embodiment, a flexible member 100 has a first end 111, a second end 112 and a body portion 110 defined therebetween. The body portion 110 has one or more magnetic sections 120. The body portion 110 may have one or more non-magnetic sections 122. The one or more magnetic sections 120 and one or more non-magnetic sections 122 alternate along the length of the body portion 110, which is defined between the first end 111 and the second end 112.

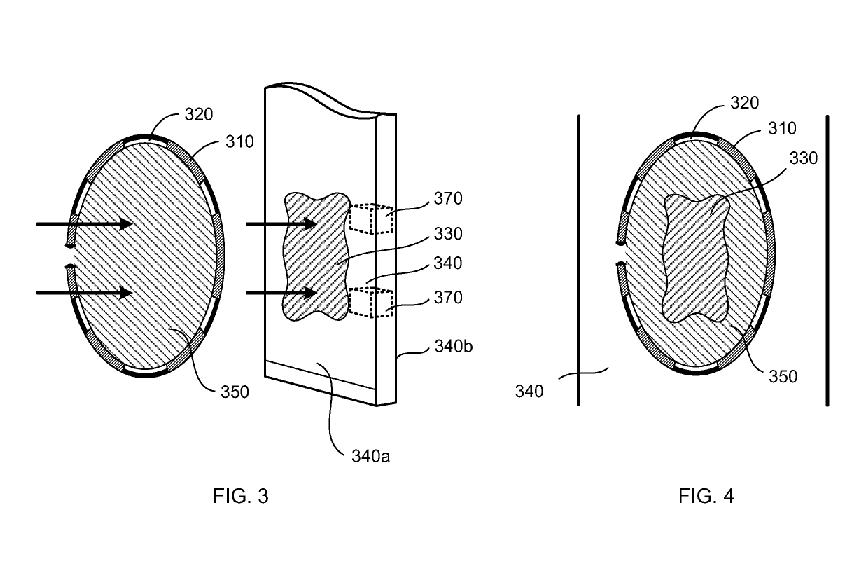

FIG. 2 shows schematically a flexible member 200 according to in another embodiment of the present invention. The flexible member 200 has a first end 211, a second end 212 and a body portion 210 defined therebetween. The body portion 210 having one magnetic section 220 that continuously extends through the length of the body portion 210, which is defined between a first end 211 and a second end 212.

The flexible member 100/200 is formed in a ring shape configuration with a biocompatible material having a shape memory property. The biocompatible material comprises a conductive material including metal or polymer. When the flexible member 100/200 is constrained, or when a force is applied to the flexible member 100/200, the flexible member 100/200 is deformed in a pre-determined shape configuration that is substantially different from the ring shape configuration. The pre-determined shape configuration can be a straight shape configuration, or the like. When the flexible member 100/200 is unconstrained, or when no force is applied to the flexible member 100/200, the flexible member 100/200 returns to its pre-formed ring shape configuration. The flexible member may be formed in other shape configurations.

In addition, the flexible member 100/200 may have means for determining a location, and a size of the fascial defect. The determining means includes a camera, one or more sensors or the like.

The magnet member includes one or more magnets. The magnet member, in operation, is positioned in relation to the one or more one magnetic sections of the flexible member such that when the magnet member is moved, it causes the flexible member to move accordingly.

The medical device can be used to determine a location and a size of the fascial defect, and to position a surgical prosthesis on a fascial defect of a tissue wall for repairing the fascial defect thereon.

Referring to FIGS. 3 and 4, the repair of a fascial defect using the invented medical device is schematically shown according to one embodiment of the present invention. Once the location and the size of the fascial defect 330 is determined, a surgical prosthesis 350, such as mesh/tissue graft, is provided and detachably attached to a flexible member 310 having a number of magnetic sections 320 along its length within the body cavity. . The tissue wall 340 bears the fascial defect 330. A magnet member 370 is positioned over the exterior surface 340b of the tissue wall 340, which is outside the body cavity. By adjusting the position of the magnet member 370 in relation to the flexible member 310, the flexible member 310 with the attached surgical prosthesis 350 is caused to be aligned to the fascial defect 330, as shown in FIG. 4. A fixation device and/or suture device can be used to fix the surgical prosthesis 350 to the fascial defect 330. Then, the flexible member 310 is detached from the surgical prosthesis 350 and removed from the body cavity. Arrows indicate a path for positioning a flexible member with a surgical prosthesis to the fascial defect on a tissue wall, using a magnet member indicated by dashed lines.

In another embodiment, after access to the body cavity, such as abdominal cavity, has been achieved, the surgical prosthesis, like mesh/tissue graft, can be introduced via this access route. The flexible ring member can be introduced into the mesh/tissue graft via an endoscope. Once the mesh/tissue graft is completely deployed and expanded, the abdominal cavity can be desufflated allowing the magnets to “lift” the mesh/tissue graft to the abdominal wall. The abdomen in insufflated again and using the magnets, the mesh/tissue graft is position to cover the hernia defect. The mesh/tissue graft is then fixed to the anterior abdominal wall via percutaneous sutures or an endoscopic fixation device.

Practically, the sized of the surgical prosthesis is larger than the size of the fascial defect. The surgical prosthesis is formed of a biocompatible mesh material, and the biocompatible mesh material comprises a tubular surgical mesh, a skirted surgical mesh, or a biological graft tissue. The surgical prosthesis is detachably attached to the flexible member.

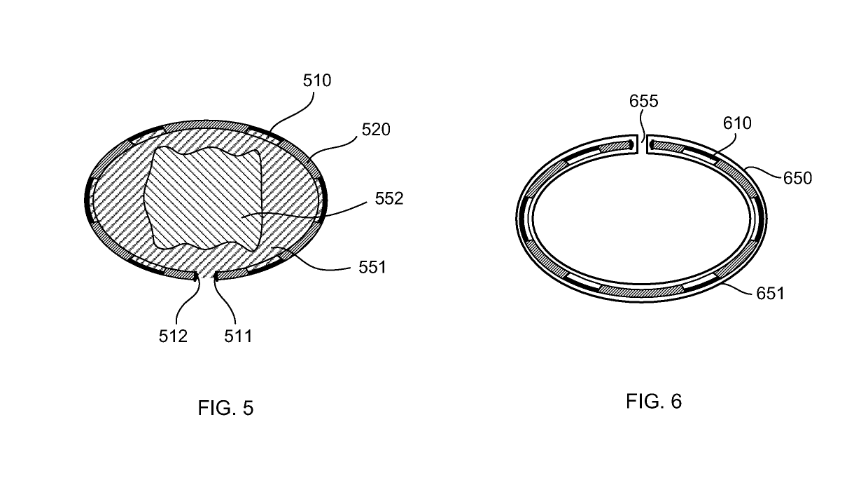

In one embodiment, as shown in FIG. 5, the surgical prosthesis 550 is formed of the skirted surgical mesh having an outer mesh portion 551 and an inner mesh portion 552, and the flexible member 510 passes into a pocket defined between the outer mesh portion 552 and the inner mesh portion 551.

In another embodiment, as shown in FIG. 6, the surgical prosthesis 650 is formed of the tubular surgical mesh in a roll 651 having an opening 655 that is defined by an inner diameter formed inside the roll 651, and the flexible member 610 passes through the opening 655 defined by the inner diameter of the roll 651.

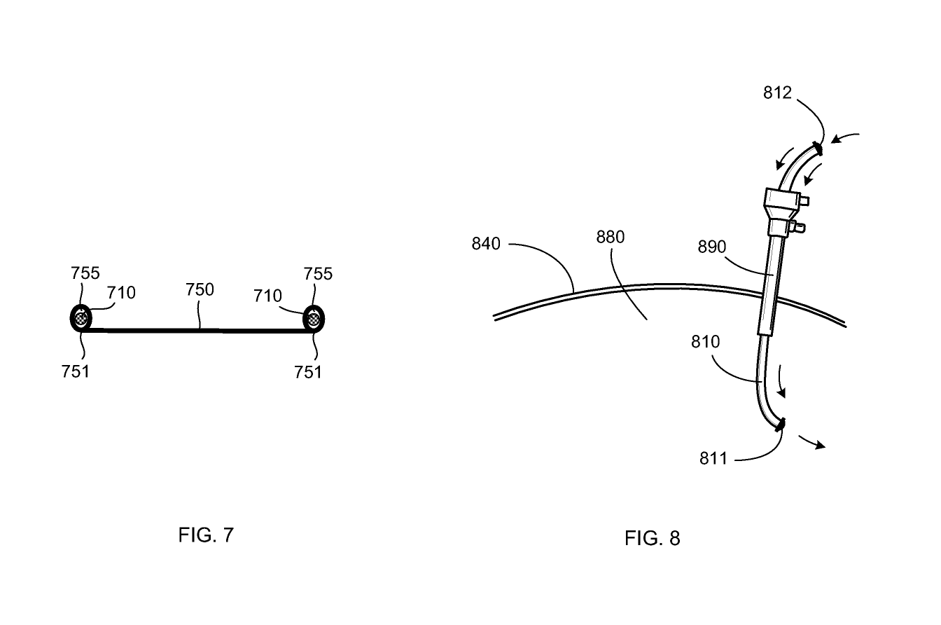

In an alternative embodiment, as shown in FIG. 7, the surgical prosthesis 750 has a peripheral edge portion 751 defining a tunnel 755 therewith, and the flexible member 710 passes through the tunnel 755 of the surgical prosthesis 750.

The flexible member can be introduced into or removed from a body cavity by a laparoscopic access device or a endoscopic access device. As shown in FIG. 8, an laparoscopic access device (trocar)890 is used to place a flexible member 810 having a first end 811 and a second end 812 into the body cavity 880 defined by the tissue wall 840 bearing a fascial defect. The diameter of the flexible member 810 is smaller than an inside diameter of the endoscope 890 for allowing the flexible member 810 to be introduced into a body cavity 880.

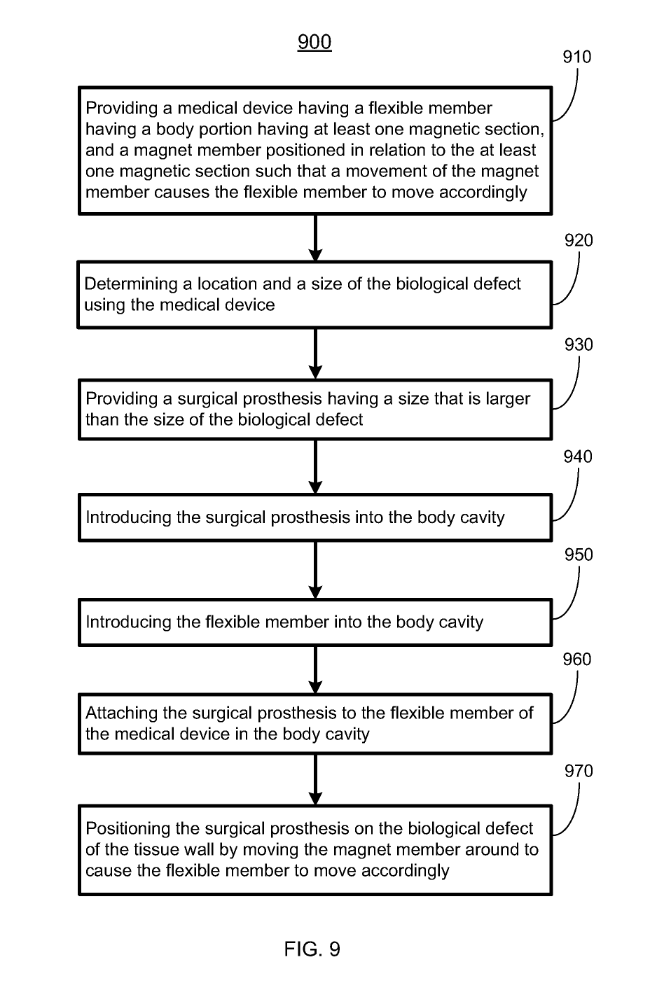

Referring to FIG. 9, a flowchart relating to a method 900 for positioning a surgical prosthesis on a fascial defect of a tissue wall of a living subject for repairing the fascial defect thereon is shown according to one embodiment of the present invention. The tissue wall defines a body cavity.

In one embodiment, the method 900 includes the following steps: at step 910, a medical device is provided. The medical device includes a flexible member having a body portion having at least one magnetic section, and a magnet member positioned in relation to the at least one magnetic section such that a movement of the magnet member causes the flexible member to move accordingly. In one embodiment, the flexible member is formed in a ring shape configuration with a biocompatible material having a shape memory property such that when constrained, the flexible member is deformed in a pre-determined shape configuration that is substantially different from the ring shape configuration, and when unconstrained, the flexible member returns to the ring shape configuration. The flexible member may have means for determining a location and a size of the fascial defect.

At step 920, a location and a size of the fascial defect is determined using the medical device. The determining step includes the steps of introducing the flexible member of the medical device into the body cavity, positioning the magnet member of the medical device outside the body cavity, adjusting the position of the magnet member of the medical device so as to move the flexible member to determine the location and the size of the fascial defect, marking the determined location of the fascial defect in the tissue wall, and removing the flexible member of the medical device from the body cavity. In one embodiment, the step of introducing the flexible member into the body cavity comprises the steps of straightening the flexible member, and introducing the straightened flexible member into the body cavity using a laparoscopic access device or a endoscopic access device.

At step 930, a surgical prosthesis having a size that is larger than the size of the fascial defect is provided. The surgical prosthesis is then introduced into the body cavity at step 940. Additionally, the flexible member is also introduced into the body cavity at step 950. Then, at step 950, the surgical prosthesis is attached to the flexible member of the medical device in the body cavity.

The surgical prosthesis is formed of a biocompatible mesh material including a tubular surgical mesh, a skirted surgical mesh, or a biological graft tissue. In one embodiment, the surgical prosthesis is formed of the skirted surgical mesh having an outer mesh portion and an inner mesh portion, and the step of attaching the surgical prosthesis to the flexible member of the medical device (step 960) includes the step of passing the flexible member into a pocket defined between the outer mesh portion and the inner mesh portion. In another embodiment, the surgical prosthesis is formed of the tubular surgical mesh is formed in a roll having an opening that defines an inner diameter of the roll, and the step of attaching the surgical prosthesis to the flexible member of the medical device (step 960) includes the step of passing the flexible member through the opening and into the inner diameter of the roll. In an alternative embodiment, the surgical prosthesis has a peripheral edge portion defining a tunnel therewith, and the step of attaching the surgical prosthesis to the flexible member of the medical device (step 960) includes the step of passing the flexible member through the tunnel.

In one embodiment, the step of attaching the surgical prosthesis to the flexible member of the medical device in the body cavity (step 960) includes the step of expanding the flexible member to stretch the surgical prosthesis into a flat configuration corresponding to the size of the fascial defect.

At step 970, the surgical prosthesis is positioned and placed on the fascial defect of the tissue wall by moving the magnet member around to cause the flexible member to move accordingly. In one embodiment, the step includes the steps of adjusting the position of the magnet member of the medical device so as to align the flexible member to the marked location of the fascial defect in the tissue wall, fixing the surgical prosthesis to the tissue wall for repairing the fascial defect, detaching the flexible member of the medical device from the surgical prosthesis, and removing the flexible member of the medical device from the body cavity. The step of fixing the surgical prosthesis to the tissue wall is performed by a fixation device and/or suture.

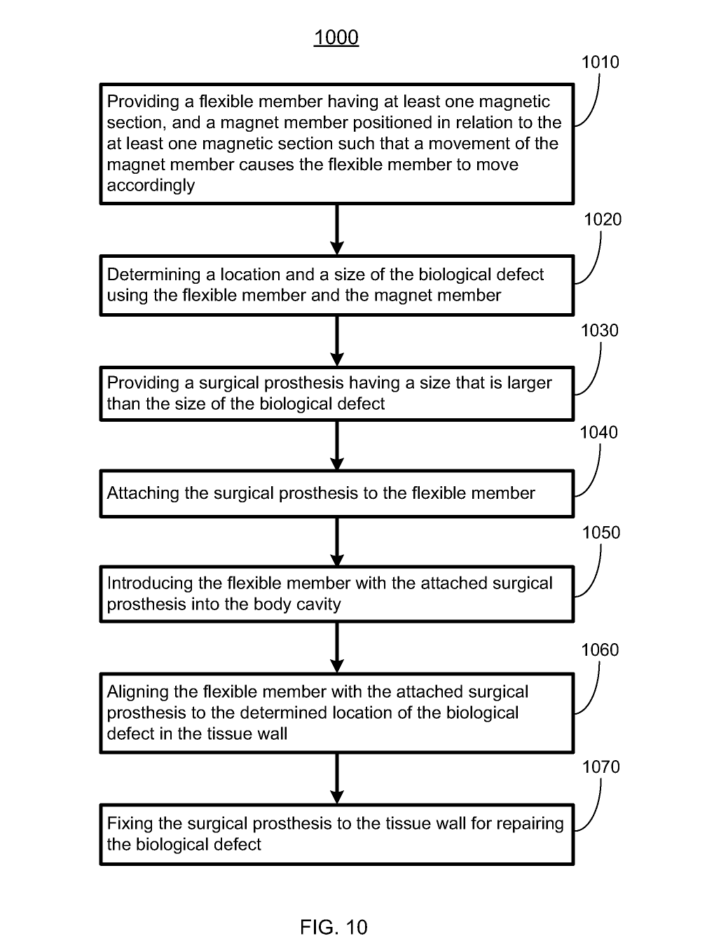

FIG. 10 shows a flowchart relating to a method 1000 for positioning a surgical prosthesis on a fascial defect of a tissue wall of a living subject for repairing the fascial defect thereon according to another embodiment of the present invention. The tissue wall defines a body cavity.

In one embodiment, the method 1000 includes steps 1010-1070:

At step 1010, a flexible member and a magnet member are provided. The flexible member has one or more magnetic sections. The magnet member in use is positioned in relation to the one or more magnetic sections such that a movement of the magnet member causes the flexible member to move accordingly.

At step 1020, a location and a size of the fascial defect is determined using the flexible member and the magnet member. The determining step comprises the steps of introducing the flexible member into the body cavity, positioning the magnet member outside the body cavity, adjusting the position of the magnet member so as to move the flexible member to determine a location and a size of the fascial defect, marking the determined location of the fascial defect in the tissue wall, and removing the flexible member from the body cavity.

At step 1030, a surgical prosthesis having a size that is larger than the size of the fascial defect is provided. The surgical prosthesis is introduced into the body cavity . The surgical prosthesis is then attached to the flexible member at step 1040.

At step 1050. In one embodiment, the step of introducing the flexible member into the body cavity is performed by a laparoscopic access device or a endoscopic access device.

At step 1060, the flexible member with the attached surgical prosthesis is aligned to the determined location of the fascial defect in the tissue wall. This step is performed by adjusting the position of the magnet member.

Then, the surgical prosthesis is fixed to the tissue wall for repairing the fascial defect at step 1070.

Furthermore, the method also includes the steps of detaching the flexible member from the surgical prosthesis, and removing the flexible member from the body cavity.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

What is claimed is:

1. A medical device for positioning a surgical prosthesis on a fascial defect of a tissue wall of a living subject for repairing the fascial defect thereon, wherein the tissue wall defines a body cavity, and wherein the fascial defect is a hernia, comprising:
(a) a flexible member having a first end, a second end and a body portion defined therebetween, the body portion having at least one magnetic section; and
(b) a magnet member positioned in relation to the at least one magnetic section such that a movement of the magnet member causes the flexible member to move accordingly,
wherein the surgical prosthesis is detachably attachable to the flexible member, and wherein the flexible member is introducible into and removable from the body cavity.

2. The medical device of claim 1, wherein in operation, the surgical prosthesis detachably attached to the flexible member is introduced into the body cavity, and the magnet member positioned outside the body cavity in relation to the flexible member is used to move the flexible member so as to position the surgical prosthesis on the fascial defect of the tissue wall.

3. The medical device of claim 2, wherein the flexible member is formed in a ring shape configuration with a biocompatible material having a shape memory property such that when constrained, the flexible member is deformed in a pre-determined shape configuration that is substantially different from the ring shape configuration, and when unconstrained, the flexible member returns to the ring shape configuration, and wherein the biocompatible material comprises a conductive material including metal or polymer.

4. The medical device of claim 3, wherein the flexible member comprises means for determining a location, and a size of the fascial defect.

5. The medical device of claim 3, wherein the at least one magnetic section continuously extends through the body portion of the flexible member.

6. The medical device of claim 2, wherein the surgical prosthesis has a size that is larger than a size of the fascial defect.

7. The medical device of claim 6, wherein the surgical prosthesis is formed of a biocompatible mesh material, and wherein the biocompatible mesh material comprises a tubular surgical mesh, a skirted surgical mesh, or a biological graft tissue.

8. The medical device of claim 7, wherein the surgical prosthesis is formed of the skirted surgical mesh having an outer mesh portion and an inner mesh portion, and wherein the flexible member passes into a pocket defined between the outer mesh portion and the inner mesh portion.

9. The medical device of claim 7, wherein the surgical prosthesis is formed of the tubular surgical mesh in a roll having an opening that defines an inner diameter of the roll, and wherein the flexible member passes through the opening and into the inner diameter of the roll.

10. The medical device of claim 7, wherein the surgical prosthesis has a peripheral edge portion defining a tunnel therewith, and wherein the flexible member passes through the tunnel.

11. A method for repairing a fascial defect in a tissue wall of a living subject, wherein the tissue wall defines a body cavity, comprising the steps of:
(a) providing a medical device having
(i) a flexible member having a body portion having at least one magnetic section; and
(ii) a magnet member positioned in relation to the at least one magnetic section such that a movement of the magnet member causes the flexible member to move accordingly;
(b) determining a location and a size of the fascial defect using the medical device;
(c) providing a surgical prosthesis having a size that is larger than the size of the fascial defect;
(d) introducing the surgical prosthesis into the body cavity;
(e) introducing the flexible member into the body cavity;
(f) attaching the surgical prosthesis to the flexible member of the medical device in the body cavity; and
(g) positioning the surgical prosthesis on the fascial defect of the tissue wall by moving the magnet member around to cause the flexible member to move accordingly.

12. The method of claim 11, wherein the flexible member is formed in a ring shape configuration with a biocompatible material having a shape memory property such that when constrained, the flexible member is deformed in a pre-determined shape configuration that is substantially different from the ring shape configuration, and when unconstrained, the flexible member returns to the ring shape configuration.

13. The method of claim 12, wherein the step of introducing the flexible member into the body cavity comprises the steps of:
(a) straightening the flexible member; and
(b) introducing the straightened flexible member into the body cavity using a laparoscopic access device or a endoscopic access device.

14. The method of claim 12, wherein the flexible member comprises means for determining a location and a size of the fascial defect.

15. The method of claim 14, the determining step comprises the steps of:
(a) introducing the flexible member of the medical device into the body cavity;
(b) positioning the magnet member of the medical device outside the body cavity in relation to the flexible member of the medical device;
(c) adjusting the position of the magnet member of the medical device so as to move the flexible member to determine a location and a size of the fascial defect;
(d) marking the determined location of the fascial defect in the tissue wall; and
(e) removing the flexible member of the medical device from the body cavity.

16. The method of claim 15, wherein the step of positioning the surgical prosthesis comprises the steps of:
(a) adjusting the position of the magnet member of the medical device so as to align the flexible member with the attached surgical prosthesis to the marked location of the fascial defect in the tissue wall;
(b) fixing the surgical prosthesis to the tissue wall for repairing the fascial defect;
(c) detaching the flexible member of the medical device from the surgical prosthesis; and
(d) removing the flexible member of the medical device from the body cavity.

17. The method of claim 16, wherein the step of fixing the surgical prosthesis to the tissue wall is performed by a fixation device and/or suture.

18. The method of claim 12, wherein the surgical prosthesis is formed of a biocompatible mesh material, and wherein the biocompatible mesh material comprises a tubular surgical mesh, a skirted surgical mesh, or a biological graft tissue.

19. The method of claim 18, wherein the surgical prosthesis is formed of the skirted surgical mesh having an outer mesh portion and an inner mesh portion, and wherein the step of attaching the surgical prosthesis to the flexible member of the medical device comprises the step of passing the flexible member into a pocket defined between the outer mesh portion and the inner mesh portion.

20. The method of claim 18, wherein the surgical prosthesis is formed of the tubular surgical mesh in a roll having an opening that defines an inner diameter of the roll, and wherein the step of attaching the surgical prosthesis to the flexible member of the medical device comprises the step of passing the flexible member through the opening and into the inner diameter of the roll.

21. The method of claim 18, wherein the surgical prosthesis has a peripheral edge portion defining a tunnel therewith, and wherein the step of attaching the surgical prosthesis to the flexible member of the medical device comprises the step of passing the flexible member through the tunnel.

22. The method of claim 12, wherein the step of attaching the surgical prosthesis to the flexible member of the medical device in the body cavity comprises the step of expanding the flexible member to stretch the surgical prosthesis into a flat configuration corresponding to the size of the fascial defect.

23. A method for repairing a fascial defect in a tissue wall of a living subject, wherein the tissue wall defines a body cavity, comprising the steps of:
(a) providing a flexible member having at least one magnetic section, and a magnet member positioned in relation to the at least one magnetic section such that a movement of the magnet member causes the flexible member to move accordingly;
(b) determining a location and a size of the fascial defect using the flexible member and the magnet member;
(c) providing a surgical prosthesis having a size that is larger than the size of the fascial defect;
(d) attaching the surgical prosthesis to the flexible member;
(e) introducing the flexible member with the attached surgical prosthesis in the body cavity;
(f) aligning the flexible member with the attached surgical prosthesis to the determined location of the fascial defect in the tissue wall; and
(g) fixing the surgical prosthesis to the tissue wall for repairing the fascial defect.

24. The method of claim 23, further comprising the steps of:
(a) detaching the flexible member from the surgical prosthesis; and
(b) removing the flexible member from the body cavity.

25. The method of claim 23, wherein the flexible member is formed in a ring shape configuration with a biocompatible material having a shape memory property such that when constrained, the flexible member is deformed in a pre-determined shape configuration that is substantially different from the ring shape configuration, and when unconstrained, the flexible member returns to the ring shape configuration.

26. The method of claim 25, wherein the step of introducing the flexible member into the body cavity is performed by a laparoscopic access device or a endoscopic access device.

27. The method of claim 25, wherein the flexible member comprises means for determining a location and a size of the fascial defect.

28. The method of claim 27, the determining step comprises the steps of:
(a) introducing the flexible member into the body cavity;
(b) positioning the magnet member outside the body cavity;
(c) adjusting the position of the magnet member so as to move the flexible member to determine a location and a size of the fascial defect;
(d) marking the determined location of the fascial defect in the tissue wall; and
(e) removing the flexible member from the body cavity.

29. A medical device for repairing the fascial defect, comprising:
(a) a flexible member having a first end, a second end and a body portion defined therebetween, the body portion having at least one magnetic section;
(b) a surgical prosthesis detachably attached to the flexible member; and
(c) a magnet member positioned in relation to the at least one magnetic section such that a movement of the magnet member causes the flexible member with the attached surgical prosthesis to move accordingly to allow the surgical prosthesis to be placed on the fascial defect.

30. A kit comprising a medical device of claim 29.
